(19) United States
(12) Patent Application Publication
(10) Pub. No.: US 2012/0238511 A1
(43) Pub. Date: Sep. 20, 2012

(54) 2-DEOXY-D-GLUCOSE FORMULATIONS FOR PREVENTION OR TREATMENT OF NEURODEGENERATIVE DISEASES

(75) Inventors: Jia Yao, Alhambra, CA (US); Roberta Diaz Brinton, Rancho Palos Verdes, CA (US)

(73) Assignee: University of Southern California

(21) Appl. No.: 13/420,418

(22) Filed: Mar. 14, 2012

Related U.S. Application Data
(60) Provisional application No. 61/452,463, filed on Mar. 14, 2011.

(51) Int. Cl. A61K 31/7004; A61P 25/28; A61P 25/00

(52) U.S. Cl. 514/23

(57) ABSTRACT

Pharmaceutical compositions containing 2-deoxy-D-glucose (2-DG) and methods of use for promoting neurological health and prevention of age-related neurodegeneration, such as Alzheimer's disease (AD), have been developed. The compositions may be formulated such that the composition, or the compound when release from the composition, crosses the blood-brain-barrier and promote neurotrophism and neuroprotection mechanisms in the brain. 2-DG can be administered alone or in combination with one or more additional therapeutic, diagnostic, and/or prophylactic agents. The compositions described herein can be administered orally, enterally, transdermally, transmucosally, intranasally or parenterally, in a dosage effective to prevent or alleviate neuronal damage, effect neuronal regeneration or sustain viability, increase expression of anti-apoptotic proteins, and/or decrease indicators of Alzheimer's Disease. The formulations can be formulated for daily, sustained, delayed or weekly/monthly administration.

### CROSS REFERENCE TO RELATED APPLICATIONS

This application claims benefit of U.S. Provisional Application No. 61/452,463 filed Mar. 14, 2011, which is hereby incorporated herein by reference in its entirety.

### STATEMENT REGARDING FEDERALLY SPONSORED RESEARCH OR DEVELOPMENT

This invention was made with Government Support under Agreement 5R01AG032236-07 awarded to Roberta Diaz Brinton by the National Institute on Aging. The Government has certain rights in the invention.

### FIELD OF THE INVENTION

This invention is in the field of 2-deoxy-D-glucose compositions for the treatment of neurological disorders such as Alzheimer's disease.

### BACKGROUND OF THE INVENTION

Millions of Americans suffer from dementia and other cognitive deficits as a result of Alzheimer's disease (AD), a neurodegenerative disease. Due to its occurrence in the brain, it is difficult to diagnose the condition and to determine its cause without dangerous brain biopsy. Scientists believe that as many as 4.5 million Americans suffer from AD. AD usually begins after age 60 and its risk goes up with age. The cause of AD is unknown and, at present, no cure has been found.

AD can only be definitively confirmed after an autopsy, which prevents early accurate diagnosis and treatment of the condition. Neuropathologically, AD is characterized by the presence of neuritic plaques, neurofibrillary tangles and neuronal loss. Doctors can typically diagnose AD correctly up to 90 percent using several tools to diagnose “probable” AD, namely, (1) questions about the person's general health, past medical problems, and ability to carry out daily activities; (2) tests of memory, problem solving, attention, counting, and language; (3) medical tests, such as tests of blood, urine, or spinal fluid; and (4) brain scans.

Postmortem brain tissues of AD victims show the presence of amyloid cores of neuritic plaques that are composed of amyloid-β protein being predominantly arranged in beta-pleated sheet configuration.

Deposition of amyloid-β protein occurs not only in individuals that have AD, but also in individuals who are undergoing the aging process. Thus, it is very critical to distinguish the AP production due to the normal aging process or to AD or other dementia-causing diseases such as DLB (dementia associated with Lewy bodies). In the normal aging process, non-compact or diffuse amyloid plaques containing less fibrillar AP are deposited primarily in the brain. In contrast, AD patients have brains that are characterized by an unanatomically widespread process of amyloid deposition and neurite plaque formation containing dense amyloid fibrils.

While many types of agents, both naturally occurring and synthetic, have been investigated for the treatment of neurological disorders, such as Alzheimer's disease, there exists a need for additional treatments which can effectively prevent or treat such neurological disorders.

Accordingly, there is a need to provide compounds for treating AD and amyloidosis-associated pathological conditions and methods of use thereof.

### SUMMARY OF THE INVENTION

Pharmaceutical compositions containing 2-deoxy-D-glucose (2-DG) and methods of use for promoting neurological health and prevention of age-related neurodegeneration, such as Alzheimer's disease (AD), have been developed. The compositions may be formulated such that the composition, or the compound released from the composition, crosses the blood-brain-barrier and promote neurotrophism and neuroprotection mechanisms in the brain. In the preferred embodiment, an effective amount of 2-DG is released to induce ketogenesis, enhance mitochondrial function, promote neurotrophic factors, reduce AD pathology, or combinations thereof.

2-DG can be administered alone or in combination with one or more additional therapeutic, diagnostic, and/or prophylactic agents. In one embodiment, 2-DG is co-administered with agents to treat neurological disorders, such as selective estrogen receptor modulators (SERMs); antineoplastic agents such as alkylating agents; antibiotics; hormonal antineoplastics and antimetabolites, such as compounds used to treat or prevent osteoporosis; vitamins; nutritional supplements; anti-oxidant agents; coenzymes; and combinations thereof. 2-DG and the one or more additional agents can be administered simultaneously in the same dosage form or different dosage forms or sequentially in different dosage forms.

The compositions described herein can be administered orally, enterally, transdermally, transmucosally, intranasally or parenterally, in a dosage effective to prevent or alleviate neuronal damage, effect neuronal regeneration or sustain viability, increase expression of anti-apoptotic proteins, and/or decrease one or more symptoms of Alzheimer's Disease. The formulations can be formulated for daily, sustained, delayed or weekly/monthly administration.

### BRIEF DESCRIPTION OF THE DRAWINGS

FIGS. 1A and 1B shows a bar graph (1A) and western blot (1B) of the relative protein expression (normalized to control) of amyloid precursor protein and the 16 kD Aβ oligomer. 2-DG drinking water reduced amyloid pathology in male 3×TgAD mice. 2-DG drinking water induced a moderate reduction in amyloid precursor protein expression level and induced a significant reduction in the 16 kD Aβ oligomer (the neurotoxic Aβ species) expression level. *, p<0.05 compared to the control (ctrl) group.

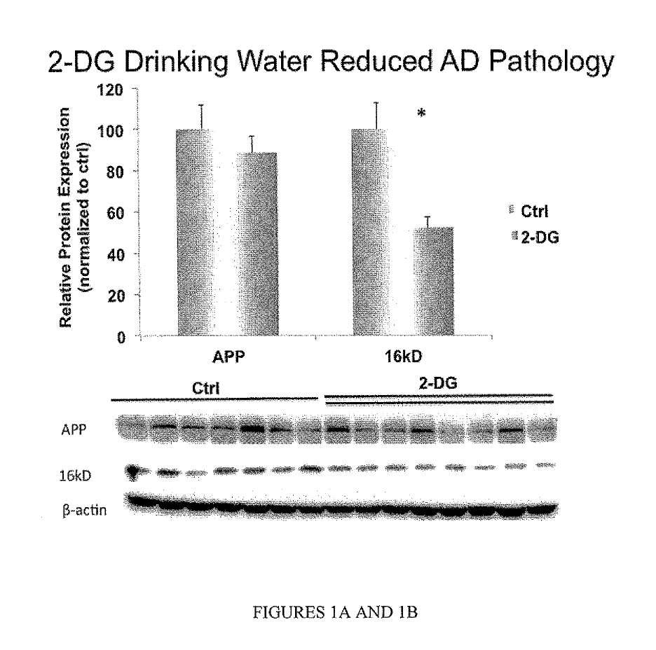

FIGS. 2A and 2B show a bar graph (2A) and western blot (2B) of the relative protein expression (normalized to control) of key enzymes involved in mitochondrial bioenergetics and energy substrate transport. 2-DG drinking water enhanced mitochondrial bioenergetics in male 3×TgAD mice. 2-DG drinking water induced significant increase in protein expression of key enzymes involved in mitochondrial bioenergetics (SCOT, αKGDH, HK2) and energy substrate transport. (MCT1, MCT2, and GLUT3). *, p<0.05 compared to the control (ctrl) group.

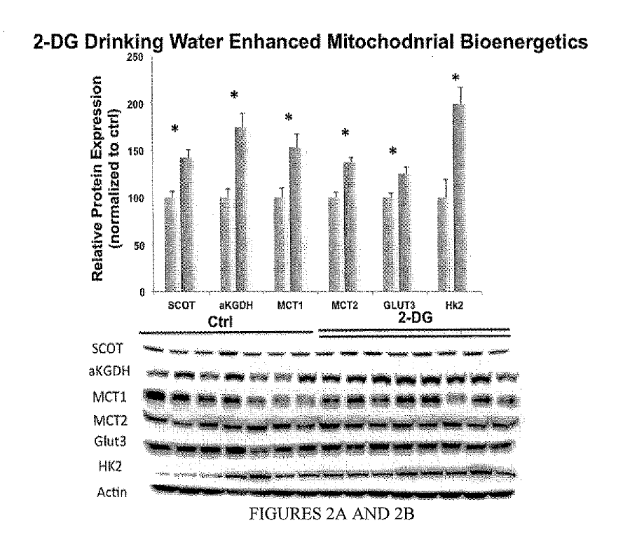

### DETAILED DESCRIPTION OF THE INVENTION

### I. Definitions

The term “effective amount” refers to any amount which results in a predetermined or desired outcome. For example, an effective amount of 2-DG may induce ketogenesis, enhance mitochondrial function, promote neurotrophic factors, reduce AD pathology, or a combination thereof. In another embodiment, an effective amount of 2-DG prevents or alleviates neuronal damage, effects neuronal regeneration or sustains viability, increases expression of anti-apoptotic proteins, and/or decreases indicators of Alzheimer's Disease.

As used herein, the term “analogue” refers to a chemical compound with a structure and function similar to that of a reference compound but differing from it in respect to a particular component, functional group, atom, etc.

As used herein, the term “derivative” refers to compounds which are formed from a parent compound by chemical reaction(s). For example, derivatives include those compounds which contain the pyran ring but differ in one or more substituents on the ring. These differences include replacement of one or more functional groups on the ring with one or more different functional groups or reacting one or more functional groups on the ring to introduce one or more substituents.

“2-deoxy-D-glucose” and “2-DG” are used interchangeably and refers to the compound 2-deoxy-D-glucose and analogues thereof.

“Amyloidosis”, as used herein, is a condition characterized by the accumulation of various insoluble, fibrillar proteins in the tissues of a patient. An amyloid deposit is formed by the aggregation of amyloid proteins, followed by the further combination of aggregates and/or amyloid proteins.

As used herein, “amylodiosis-associated pathological conditions” refers to a group of disorders caused by abnormal folding of proteins leading to fibril formation in one or more body organs, systems or soft tissues. These clumps of protein are called amyloid deposits and the accumulation of amyloid deposits causes the progressive malfunction and eventual failure of the affected organ. Normally, proteins are broken down at about the same rate as they are produced, but these unusually stable amyloid deposits are deposited more rapidly than they can be broken down. The accumulation may be localized in one organ or may be systemic such that several organs are affected. Amyloidosis causes few or no symptoms in some people, while producing severe symptoms and fatal complications in other people. The severity of the disease depends on which organs are affected by amyloid deposits. Amyloidosis is twice as common in men as in women and is more common among older people.

“Pharmaceutically acceptable salt”, as used herein, refer to 2-DG wherein the parent compound is modified by making acid or base salts thereof. Example of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

Certain compounds contained in compositions described herein may exist in particular geometric or stereoisomeric forms. The compositions described herein include all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are intended to be included as well.

If, for instance, a particular enantiomer of the compounds described herein is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Modified release dosage form: A modified release dosage form is one for which the drug release characteristics of time, course and/or location are chosen to accomplish therapeutic or convenience objectives not offered by conventional dosage forms such as solutions, ointments, or promptly dissolving dosage forms. Delayed release, extended release, and pulsatile release dosage forms and their combinations are types of modified release dosage forms.

Delayed release dosage form: A delayed release dosage form is one that releases a drug (or drugs) at a time other than promptly after administration.

Extended release dosage form: An extended release dosage form is one that allows at least a twofold reduction in dosing frequency as compared to the drug presented as a conventional dosage form (e.g. as a solution or prompt drug-releasing, conventional solid dosage form).

Pulsatile release dosage form: A pulsatile release dosage form is one that mimics a multiple dosing profile without repeated dosing and allows at least a twofold reduction in dosing frequency as compared to the drug presented as a conventional dosage form (e.g. as a solution or prompt drug-releasing, conventional solid dosage form). A pulsatile release profile is characterized by a time period of no release (lag time) or reduced release followed by rapid drug release.

### II. Compositions

Compositions containing 2-deoxyD-glucose (2-DG) are described herein. These compositions are useful for preventing and/or treating age-related cognitive decline and neurodegenerative diseases, such as Alzheimer's disease (“AD”).

A. 2-Deoxy-D-Glucose (2-DG)

The structure of 2-DG is shown below:

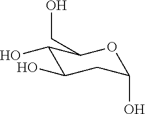

2-DG is a glucose molecule in which the 2-hydroxyl group has been replaced by hydrogen, so that it cannot undergo further glycolysis. Glucose hexokinase traps this substance in most cells (with exception of liver and kidney) so that it makes an effective marker for tissue glucose use and hexokinase activity. Many cancers have elevated glucose uptake and hexokinase levels. 2-Deoxyglucose labeled with tritium or carbon-14 has been a popular ligand for laboratory research in animal models, where distribution is assessed by tissue-slicing followed by autoradiography, sometimes in tandem with either conventional or electron microscopy.

2-DG is taken up by the glucose transporters of the cell. Therefore, cells with higher glucose uptake, for example, tumor cells, have also a higher uptake of 2-DG. Since 2-DG hampers cell growth, its use as a tumor therapeutic has been suggested, and in fact, 2-DG is in clinical trials for the treatment of tumors. However, it is not completely clear how 2-DG inhibits cell growth. The fact that glycolysis is inhibited by 2-DG, seems not to be sufficient to explain why 2-DG treated cells stop growing.

Work on the ketogenic diet as a treatment for epilepsy has investigated the role of glycolysis in the disease. 2-Deoxyglucose has been proposed to be a mimic for the ketogenic diet and has been hypothesized as a possible treatment for epilepsy. It has been suggested that 2-DG works, in part, by decreasing the expression of Brain-derived neurotrophic factor (BDNF).

The compounds, or analogues or derivatives thereof, can be used in the form of the free acid or free base or salts derived from inorganic or organic acids. These salts include, but are not limited to, the following: acetate, adipate, alginate, citrate, aspartate, benzoate, benzenesulfonate, bisulfate, butyrate, camphorate, camphorsulfonate, digluconate, cyclopentanepro-pionate, dodecylsulfate, ethanesulfonate, glucoheptanoate, glycerophosphate, hemi-sulfate, heptanoate, hexamate, fumarate, hydrochloride, hydrobromide, hydroiodide, 2-hydroxyethanesulfonate, lactate, maleate, methanesulfonate, nicotinate, 2-napthalenesulfanate, oxalate, pamoate, pectinate, sulfate, 3-phenylpropionate, picrate, pivalate, propionate, succinate, tartrate, thiocyanate, p-toluenesulfonate and undecanoate. Also, any basic nitrogen-containing groups can be quaternized with agents such as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Wafer or oil-soluble or dispersible products are thereby obtained.

Examples of acids which may be employed to form pharmaceutically acceptable acid addition salts include such inorganic acids as hydrochloric acid, sulfuric acid, and phosphoric acid, and organic acids such as oxalic acid, maleic acid, succinic acid and citric acid, Basic addition salts can be prepared in situ during the final isolation and purification of the compounds, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, and aluminum salts, as well as non-toxic ammonium, quaternary ammonium, and mine cations, including, but not limited to, ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, and piperazine.

Appropriate carriers can be added that assist the compounds to cross the blood-brain-barrier.

B. Additional Active Agents

While the compounds can be administered as the sole active pharmaceutical agent, they can also be used in combination with one or more other compounds as described herein, and/or in combination with other agents used in the treatment and/or prevention of neurological disorders. Alternatively, the compounds can be administered sequentially with one or more such agents to provide sustained therapeutic and prophylactic effects. Suitable agents include, but are not limited to, selective estrogen receptor modulators (SERMs), particularly those SERMs specific for estrogen receptor β as well as traditional estrogen agonists and antagonists.

Representative agents useful in combination with the compounds for the treatment of neurological disorders include, but are not limited to, tamoxifen, 4-hydroxytamoxifen, raloxifene, toremifene, droloxifene, TAT-59, idoxifene, RU 58,688, EM 139, ICI 164,384, ICI 182,780, clomiphene, MER-25, DES, nafoxidene, CP-336,156, GW5638, LY 139481, LY353581, zuclomiphene, enclomiphene, ethamoxytriphetol, delmadinone acetate, bisphosphonate. Other agents that can be combined with one or more of the compounds include aromatase inhibitors such as, but not limited to, 4-hydroxymdrostenedione, plomestane, exemestane, aminogluethimide, rogletimide, fadrozole, vorozole, letrozole, and anastrozole.

Still other agents useful in combination with the compounds described herein include, but are not limited to antineoplastic agents, such as alkylating agents, antibiotics, hormonal antineoplastics and antimetabolites. An example includes the compounds used to treat or prevent osteoporosis. Other ingredients include vitamins, nutritional supplements, anti-oxidant agents, coenzymes, etc.

The additional active agents may generally be employed in therapeutic amounts as indicated in the PHYSICIANS' DESK REFERENCE (PDR) 53rd Edition (2003), or such therapeutically useful amounts as would be known to one of ordinary skill in the art. The compounds and the other therapeutically active agents can be administered at the recommended maximum clinical dosage or at lower doses. Dosage levels of the active compounds in the compositions may be varied to obtain a desired therapeutic response depending on the route of administration, severity of the disease and the response of the patient. The combination can be administered as separate compositions or as a single dosage form containing all agents. When administered as a combination, the therapeutic agents can be formulated as separate compositions that are given at the same time or different times, or the therapeutic agents can be given as a single composition.

C. Pharmaceutical Compositions

The compounds can be combined with one or more pharmaceutically acceptable carriers, additives, and/or excipient for oral, enteral, transdermal, transmucosal, intranasal, or parenteral administration. The compounds can also be administered via a transdermal patch; a depo; vaginally or rectally using a topical carrier such as a gel, lotion, ointment, liposomal formulation, suspension, foam, spray or suppository; via the pulmonary or nasal route; or buccally or sublingual via the mucosal membranes of the mouth. The carriers, additives, and/or excipients are all components present in the pharmaceutical formulation other than the active ingredient or ingredients. As generally used herein “carrier” includes, but is not limited to, diluents, binders, lubricants, disintegrators, fillers, surfactants, pH modifying agents, preservatives, antioxidants, solubility enhancers, and coating compositions.

Carrier also includes all components of coating compositions which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Excipients for oral formulation are known to those skilled in the art, as discussed briefly below, and can be used to provide immediate, sustained, delayed, pulsed release, and combinations thereof. For parenteral administration, the compounds may be dissolved or suspended in saline, sterile water or phosphate buffered saline, or a suitable oil for injection intravenously (iv), intramuscularly (im), subcutaneously (subcu), infrasternal, infusion, or intraperitoneal (ip).

Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-.beta.-cyclodextrin, polyvinylpyrrollidone, low melting waxes, and ion exchange resins, as well as combinations of any two or more thereof. Other suitable pharmaceutically acceptable excipients are described in Remington's Pharmaceutical Sciences, Mack Pub. Co., New Jersey (1991).

Pharmaceutical compositions containing 2-DG or analogues or derivatives thereof, may be in any form suitable for the intended method of administration, including, for example, a solution, a suspension, or an emulsion. Liquid carriers are typically used in preparing solutions, suspensions, and emulsions. Liquid carriers contemplated for use include, for example, water, saline, pharmaceutically acceptable organic solvent(s), pharmaceutically acceptable oils or fats, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, surfactants, or stabilizers. Suitable organic solvents include, for example, monohydric alcohols, such as ethanol, and polyhydric alcohols, such as glycols. Suitable oils include, for example, soybean oil, coconut oil, olive oil, safflower oil, cottonseed oil. For parenteral administration, the carrier can also be an oily ester such as ethyl oleate, isopropyl myristate. Compositions may also be in the form of microparticles, microcapsules, liposomal encapsulates, as well as combinations of any two or more thereof.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amounts of nontoxic auxiliary substances such as wetting or emulsifying agents, dyes, pH buffering agents, or preservatives.

The compounds may be administered orally, parenterally, sublingually, by inhalation spray, rectally, vaginally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water; Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid can be useful in the preparation of injectables.

Suppositories for rectal or vaginal administration of the drug can be prepared by mixing the drug with a suitable nonirritating excipient such as cocoa butter and polyethylene glycols that are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may, be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound, stabilizers, preservatives, excipients. The preferred lipids are the phospholipids and phosphatidyl cholines (lecithins), both natural and synthetic. Methods to form liposomes are known in the art.

Transdermal patches are well known for delivery of nicotine, nitroglycerin and birth control. These can be utilized with these formulations as well. Depos that are implanted under the skin or ip can also be used, similarly to the manner of delivering birth control.

Appropriate carriers can be incorporated that assist the compounds to cross the blood-brain-barrier.

1. Modified Release Formulations

The compounds described herein can be formulated for modified release, such as immediate release, sustained release, delayed release, pulsatile release, and combinations thereof. Modified release dosage forms can be prepared by techniques known in the art and include incorporation of the compound(s) described herein into a modified release matrix which contains one or more materials that provide modified release. Alternatively, dosage forms can be coated with one or more materials to provide the desired modified release. In yet another embodiment, the compound(s) can be incorporated into micro- or nanoparticles prepared from one or more materials that provide modified release.

Suitable modified release materials are known in the art and include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides. Additionally, the coating material may contain conventional carriers such as plasticizers, pigments, colorants, glidants, stabilization agents, pore formers and surfactants.

Excipients for oral formulation are known to those skilled in the art, as discussed briefly above, and can be used in addition to immediate, sustained, delayed, and/or pulsatile release materials. For parenteral administration of modified release compositions, such as micro- and/or nanoparticles, the compounds may be dissolved or suspended in saline, sterile water or phosphate buffered saline, or a suitable oil for injection intravenously (iv), intramuscularly (im), subcutaneously (subcu), intrasternal, infusion, or intraperitoneal (ip).

### III. Methods of Administration

Compounds can be administered in a variety of ways including enteral, parenteral, pulmonary, nasal, mucosal and other topical or local routes of administration. For example, suitable modes of administration include oral, subcutaneous, transdermal, transmucosal, iontophotetic, intravenous, intramuscular, intraperitoneal, intranasal, subdural, rectal, vaginal and inhalation.

2-DG treatment induces ketogenesis, enhances mitochondrial function, promotes neurotrophic factors, and reduces AD like pathology. Data presented in the Examples indicate a therapeutic strategy to regulate brain glucose metabolism and prevents or delays the progression of AD.

An effective amount of the compound or composition is administered to treat and/or prevent a neurological disorder in a human or animal subject, particularly Alzheimer's disease (AD). For example, an effective amount of the compound or composition may induce ketogenesis, enhance mitochondrial function, promote neurotrophic factors, and/or reduce AD-like pathology, such as the reduction of amyloid precursor protein (APP). In one embodiment, the administration of 2-DG reduces the amount of APP by at least 10%, 15%, 20%, 25%, 30%, 35%, 40%, 45%, 50%, 55%, 60%, 65%, 70%, 75%, 80%, 85%, 90%, or greater compared to a suitable control. The range 10-90% includes all values between 10 and 90%.

Effective amounts of the compounds generally include any amount sufficient to detect, prevent, and/or alleviate symptoms in a subject afflicted with a neurological disorder. The effective amount may also be determined based on when the compounds are administered.

In one embodiment, the dosage is from about 0.01 mg/kg to 250 mg/kg, preferably from 0.01 mg/kg to 100 mg/kg, preferably from 0.01 mg/kg 50 mg/kg, more preferably from 0.01 mg/kg to 25 mg/kg, 0.01 mg/kg to 15 mg/kg, more preferably from 0.01 mg to 10 mg/kg, most preferably from 0.1 mg/kg to 10 mg/kg.

The compositions described herein can be administered once a day or more than once a day, such as twice a day, three times a day, four times a day or more. The compositions can be administered for a period of at least one week, two weeks, three weeks, four weeks, one month, two months, three months, four months, five months, six months, seven months, eight months, nine months, ten months, eleven months, one year, 18 months, two years, 30 months, three years, five years, seven years, ten years or longer.

### IV. Kits

Kits may be provided which contain the formulation to be administered. The formulation may be administered once a day or more than once a day. The formulation can be administered orally, enterally, parenterally, or topically. The kits typically contain the active agent(s) to be administered, excipients and carriers, and instructions for administration of the formulation. The kits may also contain equipment/devices used to administer the formulation, such as syringes.

The present invention will be further understood by reference to the following non-limiting examples.

### EXAMPLES

### Example 1

2-deoxy-D-glucose Diet Induces Ketogenesis, Enhances Mitochondrial Function, and Reduces Alzheimer's-Like Pathology in Triple Transgenic Alzheimer's Mouse Model

It has been shown that mitochondrial bioenergetic deficits precede Alzheimer's disease (AD) pathology in the triple transgenic AD (3×Tg-AD) mouse model. Both basic science and clinical studies indicated that prior to the onset or diagnosis of AD, there is a shift in brain metabolic profile from glucose-driven metabolism towards ketogenic phenotype. In the current study, the impact of 2-deoxy-D-glucose (2-DG), a compound known to regulate glucose metabolism and induce ketogenesis, on both brain bioenergetics and AD pathology was evaluated.

Materials and Methods

3×Tg-AD female at 6 month were fed with either a regular diet (AIN-93G) or diet containing 0.04% 2-DG for 7 weeks.

Results

Serum ketone levels as well as hippocampal expression of enzymes involved in ketone utilization were significantly increased with the 2-DG diet in 3×TG-AD mice. More importantly, compared to the control group, the 2-DG diet group significantly reduced AD like amyloid pathology, as manifested by decreased amyloid precursor protein (APP) and amyloid oligomer levels. Immuno-blot assays revealed increased ADAM 10 levels and decreased PS1 levels whereas BACE1 expression was not changed, suggesting a coordinated mechanism of 2-DG to simultaneous active non-amyloidogenic alpha secretase pathway while inhibiting amyloidogenic gamma secretase pathway. In addition, compared to the control diet, the 2-DG diet significantly increased the expression of brain-derived neurotrophic factors (BDNF) and increased the dendritic spine density in the hippocampal area. Additional low-density gene array (LDA array) analyses further confirmed findings that 2-DG decreased amyloid production and reduced Tau phosphorylation.

### Example 2

### Development of 2-deoxy-D-glucose Formulations

Not only is a diet formulation efficient for reducing Alzheimer's disease pathology, but a drinking water 2-DG formulation can also affect Alzheimer's disease pathology.

Materials and Methods

A drinking water 2-DG formulation was tested in a male triple transgenic mouse model of Alzheimer's disease.

Results

2-DG drinking water reduced AD pathology and promoted mitochondrial bioenergetics. The magnitude of efficacy of 2-DG drinking water is slightly lower than the 2-DG diet formulation, which can be due to compensatory increase in food intake.

Both the diet and the drinking water 2-DG formulations exhibited significant efficacy in reducing Alzheimer's pathology while simultaneously increasing indicators of bioenergetic capacity in brain.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A pharmaceutical composition comprising 2-deoxy-D-glucose and a pharmaceutically acceptable carrier for enteral or topical administration to an individual in need thereof.

2. The composition of claim 1 in a dosage unit for administration to a human, wherein the 2-deoxy-D-glucose is present in an amount effective to induce ketogenesis, enhance mitochondrial function, promote neurotrophic factors, or reduce AD pathology, and a combination thereof.

3. The composition of claim 2, wherein the amount of 2-deoxy-D-glucose is effective to decrease the amount of amyloid precursor protein in the brain.

4. The composition of claim 1, wherein the carrier is suitable for oral administration.

5. The composition of claim 4, wherein the carrier is suitable for topical administration.

6. The composition of claim 1, further comprising one or more additional therapeutic, prophylactic, and/or diagnostic agents selected from the group consisting of agents to treat neurological disorders; selective estrogen receptor modulators; estrogen agonists; estrogen antagonists; antineoplastic agents; antibiotics; hormonal antineoplastics and antimetabolites; vitamins; nutritional supplements; antioxidant agents; and coenzymes.

7. The composition of claim 1, wherein the composition is formulated for modified release.

8. The composition of claim 7, wherein modified release is selected from the group consisting of immediate release, delayed release, sustained release, pulsatile release, and combinations thereof.

9. A method for alleviating a symptom of a neurological disorder, comprising administering to a human a pharmaceutical composition comprising 2-deoxy-D-glucose and a pharmaceutically acceptable carrier for administration to an individual in need thereof.

10. The method of claim 9 wherein the 2-deoxy-D-glucose is present in an amount effective to induce ketogenesis, enhance mitochondrial function, promote neurotrophic factors, or reduce a symptom of Alzheimers disease.

11. The method of claim 9, wherein the amount of 2-deoxy-D-glucose is effective to decrease the amount of amyloid precursor protein in the brain.

12. The method of claim 9, wherein the composition is orally administered.

13. The method of claim 9, wherein the composition is topically administered.

14. The method of claim 9, further comprising administering one or more additional therapeutic, prophylactic, and/or diagnostic agents selected from the group consisting of agents to treat neurological disorders; selective estrogen receptor modulators; estrogen agonists; estrogen antagonists; antineoplastic agents; antibiotics; hormonal antineoplastics and antimetabolites; vitamins; nutritional supplements; anti-oxidant agents; and coenzymes.

15. The method of claim 9, wherein the composition is formulated for modified release.

16. The method of claim 15, wherein modified release is selected from the group consisting of immediate release, delayed release, sustained release, pulsatile release, and combinations thereof.

17. The method of claim 10, wherein the neurological disorder is Alzheimer's disease.

18. The method of claim 9, wherein the composition is administered once a day, twice a day, three times a day, or four times a day.

19. The method of claim 9, wherein the composition is administered for a period of at least one week, two weeks, four weeks, one month, two months, three months, four months, six months, eight months, or one year.
